(19) United States
(12) Patent Application Publication
(10) Pub. No.: US 2026/0108726 A1
(43) Pub. Date: Apr. 23, 2026

(54) IMPLANTABLE DEVICE CONTAINING ULTRASOUND-RESPONSIVE METAMATERIALS

(71) Applicant: ENERGY MINING Co., Ltd., Suwon-si (KR)

(72) Inventors: Youngwook CHUNG, Suwon-si (KR); Jeongun CHOE, Yongin-si (KR); Joonha HWANG, Suwon-si (KR); Jangmook JEONG, Suwon-si (KR); Hyunmoon PARK, Gunpo-si (KR)

(73) Assignee: ENERGY MINING Co., Ltd., Suwon-si (KR)

(21) Appl. No.: 19/020,756

(22) Filed: Jan. 14, 2025

(30) Foreign Application Priority Data
Oct. 18, 2024 (KR) 10-2024-0143376

(51) Int. Cl. A61N 1/05; A61N 1/36; A61N 1/378

(52) U.S. Cl. CPC A61N 1/0551; A61N 1/3605; A61N 1/3787

(57) ABSTRACT

According to one embodiment, an implantable device may include a substrate having a dielectric layer and the first electrode layer disposed thereon; a first dielectric-metal-dielectric (DMD) layer disposed on the substrate and having a charged layer and a membrane disposed symmetrically to each other with reference to an electrode layer; a metal-dielectric-metal (MDM) layer disposed on the first DMD layer and having an electrode layer symmetrically disposed with reference to the membrane; a second DMD layer disposed on the MDM layer; a sealing layer disposed on the second DMD layer; and a metamaterial layer disposed on the sealing layer to press the sealing layer.

### CROSS-REFERENCE TO RELATED APPLICATION

The present application claims the benefit of Korean Application No. 10-2024-0143376 filed in the Korean Intellectual Property Office on Oct. 18, 2024, the entire contents of which are incorporated herein by reference.

### BACKGROUND OF THE DISCLOSURE

### Field of the Disclosure

The present disclosure relates to an implantable device, and more particularly, to an implantable device containing an ultrasound-responsive metamaterial.

### Background of the Related Art

As medical technology advances, there is an increasing demand for miniaturization of implantable medical devices. Implantable medical device in the related art often cause a biological burden on patients' bodies due to a size and a weight, and to address this, miniaturization has become an essential direction for development. Miniaturized medical devices may offer reduced invasiveness when implanted into a body, shorten patient recovery time, and reduce long-term side effects in the body. In addition, to solve a problem of having to use an external power source for operation of the implantable medical devices, a need for wireless power transfer technology has emerged.

As one of technologies for resolving this need, ultrasound-driven triboelectric generators are drawing attention. Unlike an electromagnetic induction method in the related art, wireless power transfer technology using ultrasound waves does not need a resonance design. Thus, power transfer may be simply and efficiently performed. In particular, a triboelectric nano-generation device is well suited to converting minute movement and pressure occurring in a body into electrical energy. This enables a stable power supply to implantable medical devices. When a power generator is driven through an ultrasound driven method, power may be produced with high efficiency in an environment in a body. Thus, limitations in a power supply method in the related art may be overcome.

Various materials may be used in ultrasound-driven triboelectric generators, and each material performs an important function in power conversion efficiency and durability. Such a device may include polymers, metal nanoparticles, or biocompatible composite materials, and is designed to minimize interaction with a body when implanted into the body. Particularly, a triboelectric nano-generation device provides high energy density despite a small size, and thus, have high potential to be used for next-generation wireless power transfer technology. This power generation technology may greatly improve autonomy of future implantable medical devices, thereby contributing to reducing a need for external power sources in a long term.

### SUMMARY

A power generation capacity (e.g., output voltage) of a triboelectric nanogenerator is determined by a size of an active area and displacement. Since a nanogenerator equipped in an implantable medical device is limited in size, there is a limitation in an increase in a power generation capacity.

Embodiments of the present disclosure are directed to addressing such needs, and provide an implantable device (e.g., a triboelectric nanogenerator) with a maximized power generation capacity.

However, one or more embodiments are only examples, and the scope of the present disclosure is not limited thereto.

According to one embodiment, an implantable device may include a substrate having a dielectric layer and the first electrode layer disposed thereon; a first dielectric-metal-dielectric (DMD) layer disposed on the substrate and having a charged layer and a membrane disposed symmetrically to each other with reference to an electrode layer; a metal-dielectric-metal (MDM) layer disposed on the first DMD layer and having an electrode layer symmetrically disposed with reference to the membrane; a second DMD layer disposed on the MDM layer; a sealing layer disposed on the second DMD layer; and a metamaterial layer disposed on the sealing layer to press the sealing layer.

According to one embodiment, the metamaterial layer may include: weight members arranged periodically to press the sealing layer; and an elastic member including a dielectric material and surrounding the weight members.

According to one embodiment, the elastic member may include a base portion having a flat plate shape and a pattern portion protruding from the base portion toward the substrate, the weight members may be respectively provided below the pattern portion, and the pattern portion may vibrate up and down due to a weight of the weight members to expand or contract.

According to one embodiment, a first spacer disposed between the substrate and the first DMD layer; a second spacer disposed between the first DMD layer and the MDM layer; a third spacer disposed between the MDM layer and the second DMD layer; and a fourth spacer arranged between the second DMD layer and the sealing layer may be further included.

According to one embodiment, the first DMD layer may include a first charged layer disposed on the first electrode layer, a first membrane disposed on the first charged layer, a second electrode layer disposed on the first membrane, a second membrane disposed on the second electrode layer, and a second charged layer disposed on the second membrane, the first charged layer may be disposed to expose at least a part of a lower portion of the first membrane, and the second charged layer may be disposed to expose at least a part of an upper portion of the second membrane.

According to one embodiment, the MDM layer may include a third electrode layer disposed on the second charged layer, a third membrane disposed on the third electrode layer, and a fourth electrode layer disposed on the third membrane, and the third membrane may be disposed to have at least a part of a lower portion exposed by the third electrode layer and at least a part of an upper portion exposed by the fourth electrode layer.

According to one embodiment, the second DMD layer may include a third charged layer disposed on the fourth electrode layer, a fourth membrane disposed on the third charged layer, a fifth electrode layer disposed on the fourth membrane, a fifth membrane disposed on the fifth electrode layer, and a fourth charged layer disposed on the fifth membrane, the third charged layer may be disposed to expose at least a part of a lower portion of the fourth membrane, and the fourth charged layer may be disposed to expose at least a part of an upper portion of the fifth membrane.

According to one embodiment, the sealing layer may include a sixth electrode layer disposed on the fourth charged layer and a sixth membrane disposed on the sixth electrode layer, the sixth electrode layer may be disposed to expose at least a part of a lower portion of the sixth membrane, and the first electrode layer may be disposed to expose at least a part of an upper portion of the dielectric layer.

According to one embodiment, the first spacer may include a first-first electrode layer disposed to cover at least a part of an upper portion of the first electrode layer and a dielectric layer disposed to have a same area as an area of the first-first electrode layer, and the first-first electrode layer may be in contact with and electrically connected to the first electrode layer. In addition, the second spacer may include a third-first electrode layer disposed to cover at least a part of a lower portion of the third electrode layer and a dielectric layer disposed to have a same area as an area of the third-first electrode layer, and the third-first electrode layer may be in contact with and electrically connected to the third electrode layer.

According to one embodiment, the third spacer may include a fourth-first electrode layer disposed to cover at least a part of an upper portion of the fourth electrode layer and a dielectric layer disposed to have a same area as an area of the fourth-first electrode layer, and the fourth-first electrode layer may be in contact with and electrically connected to the fourth electrode layer. In addition, the fourth spacer may include a sixth-first electrode layer disposed to cover at least a part of a lower portion of the sixth electrode layer and a dielectric layer disposed to have a same area as an area of the sixth-first electrode layer, and the sixth-first electrode layer may be in contact with and electrically connected to the sixth electrode layer.

In addition to those described above, other aspects, features and effects will become apparent from the following drawings, claims, and detailed descriptions of the present disclosure.

An implantable device according to the present disclosure may include a triboelectric nanogenerator. The implantable device includes layers stacked to increase an active area, and a metamaterial layer for allowing sufficient vibration of the stacked layers (e.g. (for securing sufficient displacement). As a result, the implantable device according to the present disclosure may be manufactured in a size to be capable of being implanted into a body while increasing a power generation capacity.

In addition, spacers according to the present disclosure are configured to be capable of being bonded to other layers, and thus, has an advantage in a process of manufacturing a triboelectric nanogenerator.

### BRIEF DESCRIPTION OF THE DRAWINGS

The above and other objects, features and advantages of the present disclosure will be apparent from the following detailed description of the embodiments of the disclosure in conjunction with the accompanying drawings, in which:

FIGS. 1 to 4B are diagrams for explaining an ultrasound-based triboelectric nanogenerator;

FIGS. 5A and 5B illustrate examples of a triboelectric nanogenerator;

FIG. 6 is a schematic diagram of an implantable device containing an ultrasound-responsive metamaterial according to one embodiment;

FIG. 7 is a diagram for explaining a metamaterial layer according to one embodiment;

FIG. 8 is a diagram for explaining a cross-section of the implantable device containing an ultrasound-responsive metamaterial according to one embodiment;

FIGS. 9A to 10B are diagrams for explaining vibration of the implantable device containing an ultrasound-responsive metamaterial according to one embodiment;

FIGS. 11 and 12 are diagrams for explaining a process of manufacturing the implantable device containing an ultrasound-responsive metamaterial according to one embodiment;

FIGS. 13 to 15 are diagrams for explaining a process of manufacturing each layer according to one embodiment; and

FIGS. 16A to 16C are diagrams for explaining the implantable device containing an ultrasound-responsive metamaterial according to one embodiment.

### DETAILED DESCRIPTION

Since the present disclosure may have various modifications and several embodiments, embodiments are shown in the drawings and will be provided in the detailed description in detail. Effects and features of the present disclosure and methods of accomplishing the same may be understood more readily with reference to the following detailed description of embodiments and the accompanying drawings. However, the present disclosure is not limited to the embodiments set forth herein, and may be embodied in many different forms.

It will be understood that although the terms “first,” “second,” etc. may be used herein to describe various components, these components should not be limited by these terms. These components are only used to distinguish one component from another.

As used herein, the singular forms “a,” “an” and “the” are intended to include the plural forms as well, unless the context clearly indicates otherwise.

It will be further understood that the terms “comprises” and/or “comprising” used herein specify the presence of stated features or components, but do not preclude the presence or addition of one or more other features or components.

It will be understood that when a layer, region, or component is referred to as being “arranged on,” another layer, region, or component, it can be directly or indirectly arranged on the other layer, region, or component. That is, for example, intervening layers, regions, or components may be present.

Sizes of elements in the drawings may be exaggerated for convenience of explanation. In other words, since sizes and thicknesses of components in the drawings are arbitrarily illustrated for convenience of explanation, the following embodiments are not limited thereto.

When a certain embodiment may be implemented differently, a particular process order may be performed differently from the described order. For example, two consecutively described processes may be performed substantially at the same time or performed in an order opposite to the described order.

In the present specification, “A and/or B” refers to A or B, or A and B. In addition, “at least one of A and B” refers to A or B, or A and B.

It will be understood that when a layer, region, or component is referred to as being connected to or coupled to another layer, region, or component, it may be directly connected or coupled to the other layer, region, or component, and/or indirectly connected to the other layer, region, or component with intervening elements therebetween. For example, when a layer, region, or component is referred to as being electrically connected to or coupled to another layer, region, or component, it may be electrically directly connected or coupled to the other layer, region, or component, and/or electrically indirectly connected to the other layer, region, or component with intervening elements therebetween.

The x-axis, the y-axis and the z-axis are not limited to three axes of the rectangular coordinate system, and may be interpreted in a broader sense. For example, the x-axis, the y-axis, and the z-axis may be perpendicular to one another, or may represent different directions that are not perpendicular to one another.

As used herein, the term is intended to illustrate the embodiments but is not intended to limit the inventive concept. In this specification, the singular includes the plural unless specifically stated otherwise. It will be further understood that the terms “comprises,” “comprising,” “includes,” and/or “including,” when used herein, specify the presence of members, but do not preclude the presence or addition of one or more other members, unless otherwise specified.

A word “exemplary” used herein means “used as an example or illustration.” Any embodiment described herein as “exemplary” is not necessarily to be construed as desirable or advantageous over other embodiments.

Embodiments of the present disclosure may be described in terms of functions or blocks that perform functions. Blocks which may be referred to as ‘units’ or ‘modules’ in the present disclosure may be physically implemented by analog or digital circuits such as logic gates, integrated circuits, microprocessors, microcontrollers, memories, passive electronic components, active electronic components, optical components, hardwired circuits, and the like, and may optionally be driven by firmware and software. Additionally, a term ‘unit’ means software or hardware elements such as field programmable gate array (FPGA) or application-particular integrated circuit (ASIC), and a “unit” performs some functions. However, a “unit” is not limited to hardware or software. A “unit” may be configured to be included in a storage medium that may be addressed, or configured to play one or more processors. Accordingly, as an example, a “unit” includes elements such as software elements, object-oriented software elements, class components, or task elements, processes, functions, attributes, procedures, subroutines, segments of a program code, drivers, firmware, micro-codes, circuits, data, database, data structures, tables, arrays, or variables. Functions provided in elements or “units” may be combined into a small number of elements or “units,” or separated into additional elements or “units.”

An embodiment of the present disclosure may be implemented using at least one software program running on at least one hardware device, and capable of performing network management functions to control elements.

Spatially relative terms such as “below,” “beneath,” “lower,” “above,” and “upper” may be used to easily describe a relationship of one component with other components as illustrated in the drawings. Spatially relative terms are to be understood as a term that includes other directions of the element in use or operation in addition to the direction illustrated in the drawings. For example, in a case in which a component shown in the drawing is described as being “below” or “beneath” another member, when the component is turned upside down, the component may be placed “above” the other member. Thus, the exemplary term “below” may include both downward and upward directions. Components may be oriented in other directions, and thus, the spatially relative terms may be interpreted according to the orientation.

Unless otherwise defined, all terms (including technical and scientific terms) used herein may be used as having a meaning that can be understood in common by one of ordinary skill in the art. In addition, terms defined in a generally used dictionary are not interpreted ideally or excessively, unless otherwise defined explicitly and particularly.

Hereinafter, embodiments of the present disclosure will be described in detail with reference to the attached drawings, in which like reference numerals designate like elements and repetitive explanation thereof will be omitted.

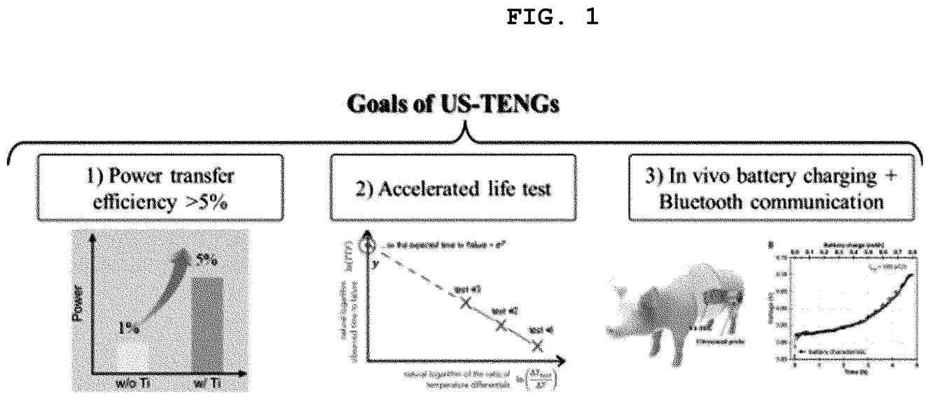

FIGS. 1 to 4B are diagrams for explaining an ultrasound-based triboelectric nanogenerator.

Referring to FIG. 1, goals of the ultrasound-based triboelectric nanogenerator (US-TENG) may be identified.

A first goal of the ultrasound-based triboelectric nanogenerator is to increase power transfer efficiency to 5% or higher. Referring to FIG. 1, power transfer efficiency is only 1% when titanium (Ti) is not used, but the first goal is to increase the power transfer efficiency to 5% by efficiently applying titanium.

A second goal is to sufficiently validate durability of a device through accelerated life testing. Referring to FIG. 1, a process of predicting expected lifespan by measuring failure time of a device according to various temperature differences is shown. The accelerated life testing is an important process of quickly checking whether the device can be used for a long time, and is an essential operation for ensuring device reliability.

A third goal is to realize in-vivo battery charging and Bluetooth communication. Referring to FIG. 1, an experiment of charging a battery in a pig is shown, and the third goal is to develop a function of transceiving data via Bluetooth communication while charging the battery. This presents various bio-application possibilities, such as a medical device, a wearable device, etc.

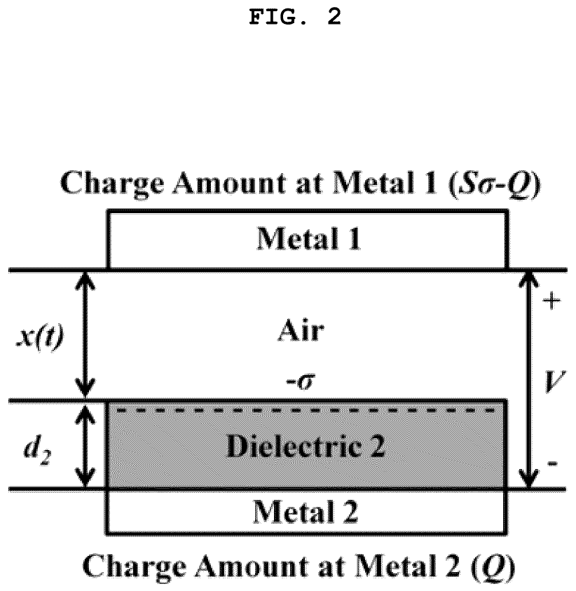

FIG. 2 is a diagram for explaining an electrical principle related to a triboelectric nanogenerator (TENG).

A generation power capacity of the triboelectric nanogenerator (TENG) may be described based on an open-circuit voltage VOC and an accumulated charge Q_{sc} in a short-circuit state. The open circuit voltage V_{OC} may be calculated using Equation 1.

$${V}_{OC} = \frac{σ x ( t )}{{ε}_{0}} \qquad [ Equation 1 ]$$

The open circuit voltage (V_{OC}) is determined based on a charge density σ, a displacement function x (t) indicating a distance between two charged layers, and a permittivity ε₀ in vacuum. Equation 1 shows that a voltage between two surfaces in the TENG is determined according to charges accumulated on the two surfaces and a distance function.

The accumulated charge Q_{sc} in the short-circuit state may be calculated using Equation 2.

$${Q}_{SC} = \frac{S σ x ( t )}{{d}_{0} + x ( t )} \qquad [ Equation 2 ]$$

In the short circuit state, the accumulated charge Q_{sc} is determined based on an area S of a surface (e.g., an active area), a charge density σ on the surface, an initial distance do, and a displacement function x (t).

As may be checked from Equations 1 and 2, the active area S and the displacement function x (t) need to be increased to increase a power generation capacity of the TENG.

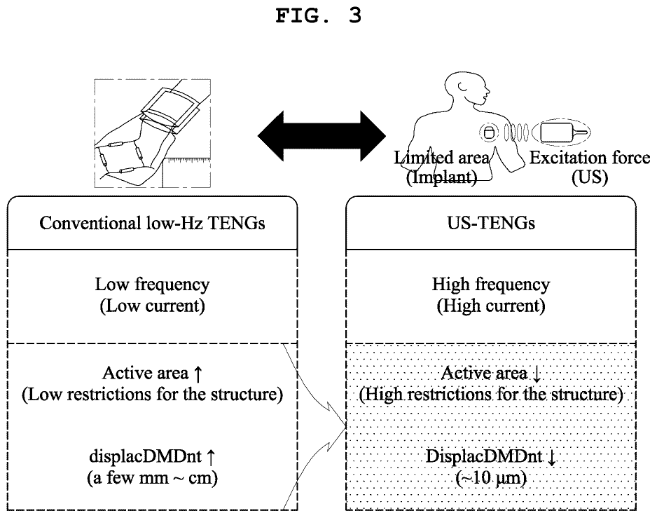

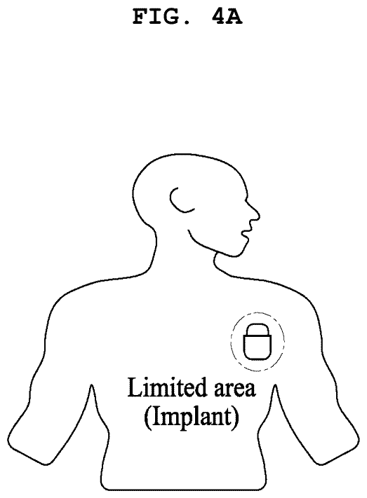

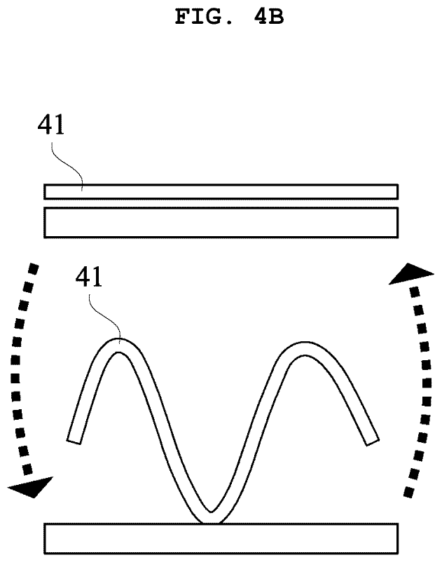

Referring to FIGS. 3 to 4B, main challenges for a US-TENG may be checked.

Referring to FIGS. 3 and 4A, a low-frequency TENG in the related art operates at a low frequency, and thus, generates low current. However, this structure has a relatively large active area and allows a large displacement of a few millimeters to a few centimeters.

On the other hand, the US-TENG may operate at high frequencies and generate high current, but has a very limited active area and significant structural restrictions due to a small size. In addition, since an allowable displacement is very small, approximately 10 μm, a range of mechanical motion is also limited. Since the US-TENG needs to operate in a miniaturized implant or a limited space, a design and structural optimization emerges as an important task to be solved.

Referring to FIG. 4B, it may be checked that a membrane 41 in the US-TENG does not vibrate integratedly and unidirectionally, but parts of the membrane 41 vibrate, respectively (e.g., multimode vibration). This may cause a substantial reduction in entire membrane displacement. In addition, due to regulations on intensity in the United States, there is a problem in that external force that may be provided to the US-TENG to increase displacement may be limited.

In conclusion, the US-TENG needs structural and mechanical optimization regarding a method of efficiently producing energy under conditions such as a limited active area, small displacement, and limited external force environment, while maintaining high frequency and high current.

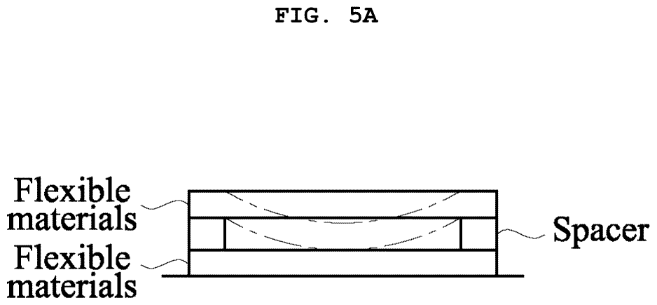

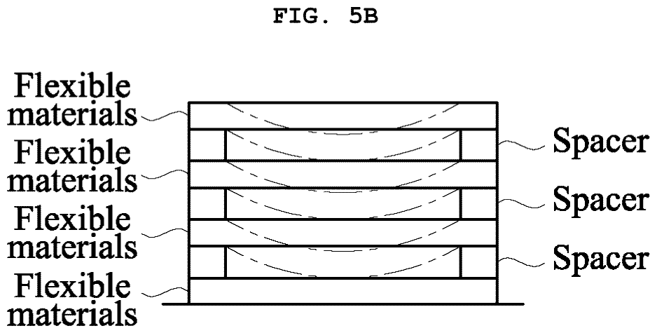

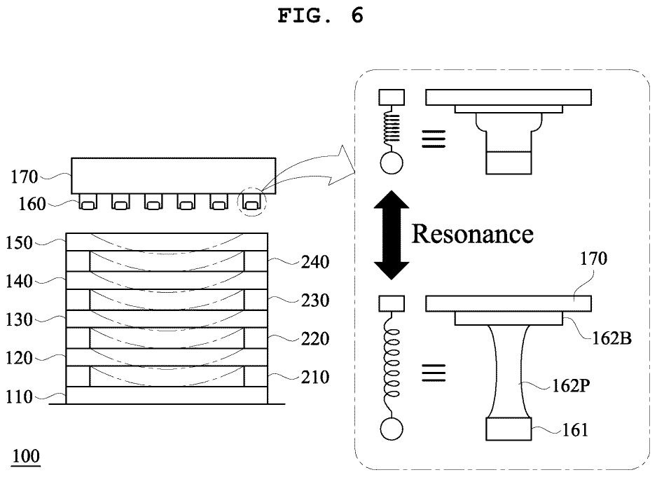

FIGS. 5A and 5B illustrate examples of a triboelectric nanogenerator.

Referring to FIG. 5A, the triboelectric nanogenerator (TENG) may include a spacer and an elastic flexible material. This allows the TENG to utilize a pushing motion according to external force. In this structure, a flexible material is deformed in response to external pressure, and an active area and displacement are increased. As the displacement increases through the flexible material, output generated by the TENG may also be enhanced.

Referring to FIG. 5B, a TENG having stacked layers may be checked. An active area in the TENG may be further increased through further stacking of layers. The flexible materials and spacer structures stacked in multiple layers may collect energy in more areas, and according to addition of the layers, output may be increased proportionally. This is a structural improvement method that enables efficient energy collection even in a limited space.

In conclusion, to improve efficiency of the TENG, advancements are being made such that displacement and an active area are increased using flexible materials, and the flexible materials are stacked in multiple layers to maximize an energy collection capability.

A TENG equipped in an implantable device in a body may have a limited power generation capacity due to limitation in a size. Generally, in a wireless power transfer system, a size of a receiver is proportional to power generation efficiency. This phenomenon is prominent in technologies having capacitive characteristics. Since the TENG also generates displacement current based on the capacitive characteristics, an active area performs as a very important function.

As described above, research on the TENG has adopted a layer stacking structure to overcome such a limitation in an active area. However, this approach may increase a size and volume of a device, thus causing a restriction in application fields in which miniaturization is essential, such as a TENG that is to be implanted inside a body.

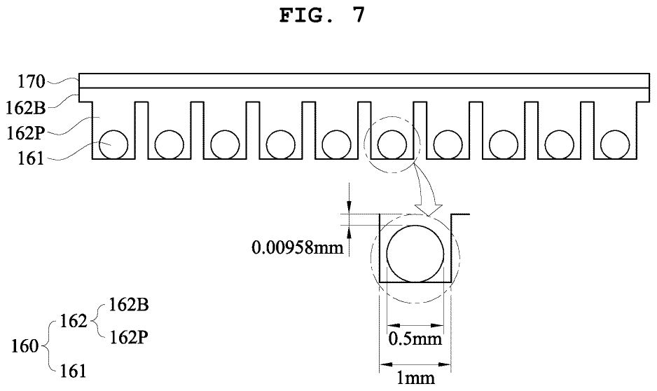

FIG. 6 is a schematic diagram of an implantable device containing an ultrasound-responsive metamaterial according to one embodiment. FIG. 7 is a diagram for explaining a metamaterial layer according to one embodiment.

Referring to FIG. 6, an implantable device 100 containing an ultrasound-responsive metamaterial according to one embodiment is shown. The implantable device 100 may include a triboelectric nanogenerator (TENG) 100. Two most important factors in a TENG include an active area and displacement. To do so, in the implantable device 100 according to one embodiment, layers 120 to 150 having a small thickness are stacked, and a metamaterial layer 160 is introduced to maximize displacement, thereby increasing efficiency of the implantable device 100.

The implantable device 100 according to one embodiment may have the layers 120 to 150 stacked on each other to expand the active area and include the metamaterial layer 160 to allow sufficient vibration of the stacked layers 120 to 150 (e.g., to secure sufficient displacement). As a result, the implantable device 100 may be manufactured in a size to be capable of being implanted into a body while increasing a power generation capacity.

In addition, The first to fourth spacers 210 to 240 in the implantable device 100 are disposed to be bonded to other layers to have an advantage in a process of manufacturing the triboelectric nanogenerator.

The implantable device 100 may include a substrate 110 on which a dielectric layer and a first electrode layer are disposed.

The implantable device 100 may include a first dielectric-metal-dielectric (DMD) layer 120 disposed on the substrate 110 and having a charged layer and a membrane disposed symmetrically to each other with reference to an electrode layer. At this time, the first spacer 210 may be placed between the substrate 110 and the first DMD layer 120.

The implantable device 100 may include an MDM layer 130 disposed on the first DMD layer 120 and having electrode layers symmetrically disposed with reference to the membrane. At this time, a second spacer 220 may be placed between the first DMD layer 120 and the MDM layer 130.

The implantable device 100 may include a second DMD layer 140 disposed on the MDM layer 130. At this time, a third spacer 230 may be placed between the MDM layer 130 and the second DMD layer 140.

The implantable device 100 may include a sealing layer 150 disposed on the second DMD layer 140. At this time, a fourth spacer 240 may be placed between the second DMD layer 140 and the sealing layer 150.

The implantable device 100 may include the metamaterial layer 160 disposed on the sealing layer 150 to press the sealing layer 150. The implantable device 100 may further include a titanium layer 170 disposed on the metamaterial layer 160.

The metamaterial layer 160 may operate like a spring as illustrated in FIG. 6. In the present disclosure, a metamaterial may refer to a structure artificially manipulated to have a particular electromagnetic or mechanical property. The metamaterial may have a property capable of controlling a path of an electromagnetic or acoustic wave, or diversely controlling absorption, reflection, or refraction. An electromagnetic property of the metamaterial may be determined mainly through a structure of repetitive arrangement in units of nanometers or micrometers.

The metamaterial layer 160 may include weight members 161 periodically arranged to press the sealing layer 150. The metamaterial layer 160 may include an elastic member 162 including a dielectric material and surrounding the weight members 161.

Hereinafter, the metamaterial layer 160 will be described further in detail with reference to FIG. 7. The elastic member 162 may include a base portion 162B having a flat plate shape and a pattern portion 162P protruding from the base portion 162B toward the substrate 110. A width and a length of the base portion 162B may correspond to a width and a length of the titanium layer 170.

The weight members 161 may be each provided below the pattern portion 162P. The pattern portion 162P may vibrate up and down due to a weight of the weight members 161 to expand or contract. FIG. 7 illustrates that the pattern portion 162P has a consistent diameter. However, the pattern portion 162P is not limited thereto, and may be implemented to have another physical structure effective to cover the weight members 161.

The weight members 161 may be configured to contain steel use stainless (SUS) 304 and have a density σf 7.93 g/cm³ and a diameter of 0.5 mm. At this time, when the weight members 161 are implemented to have a shape of a sphere, a mass thereof may be calculated using Equation 3.

$$m = \frac{4 π}{3} \cdot {( 0.025 cm )}^{3} \cdot 7.93 g / {cm}^{3} = 5.1902 \times {10}^{- 4} g \qquad [ Equation 3 ]$$

The pattern portion 162P may be configured to contain silicon, have an elastic modulus of 1 MPa, and have a width of 1 mm and a length of 0.00958 m. At this time, a spring constant k and a natural frequency f₀ of the pattern portion 162P may be calculated using Equation 4.

$$k = E \frac{{A}_{x}}{L} = \text{?} ( 1 MPa ) \frac{{π ( 0. \text{?} mm )}^{2}}{0.00958 \text{?} mm} = \text{?} 8 , 196 \text{?} Pa \qquad [ Equation 4 ]$$

$${f}_{0} = \frac{1}{2 π} \sqrt{\frac{k}{m}} = \frac{1}{2 π} \sqrt{\frac{8 , 19 \text{?} Pa}{5.19 \times 1 \text{?} kg}} = 20000 \text{?} Hz \text{?}$$

$$\text{?} \text{indicates text missing or illegible when filed}$$

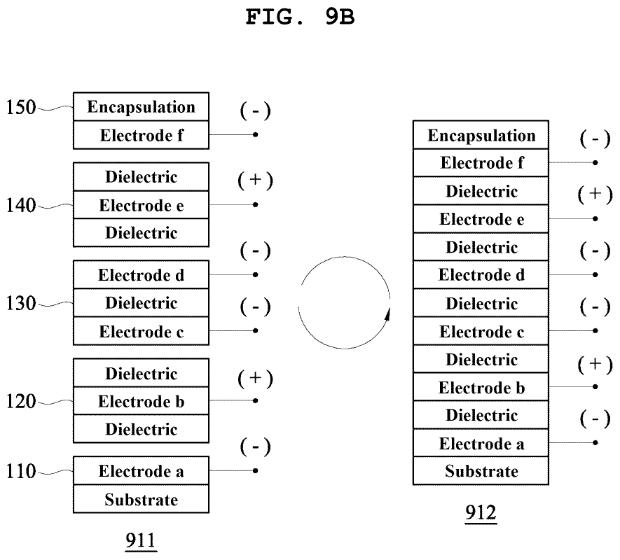

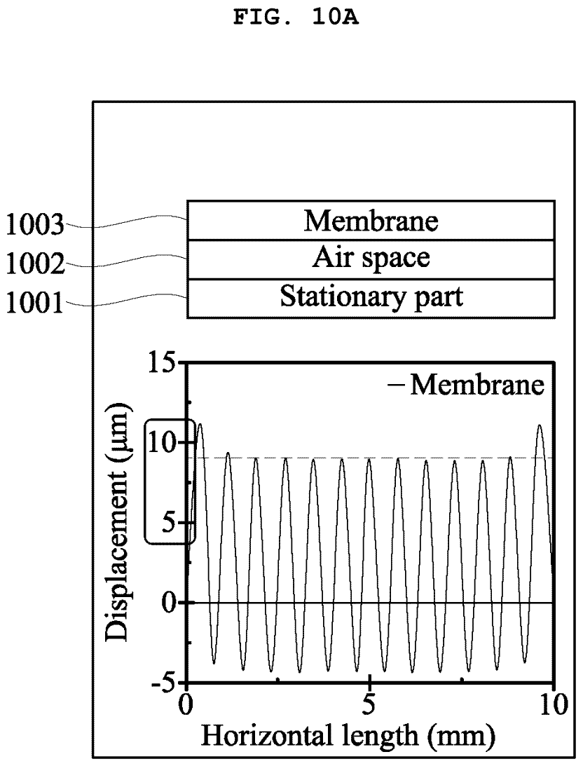

As may be understood from FIG. 7 and Equations 3 and 4, the metamaterial layer 160 may function as a spring structure. When an ultrasound wave is applied to the metamaterial layer 160, an upper surface of the sealing layer 150 may be pressed through a vibration motion caused by the application of the ultrasound waves. That is, the implantable device 100 may include the metamaterial layer 160 to allow the layers 120 to 150 stacked on each other to sufficiently vibrate (e.g., for displacement) (e.g., see FIGS. 9A and 9B, etc.).

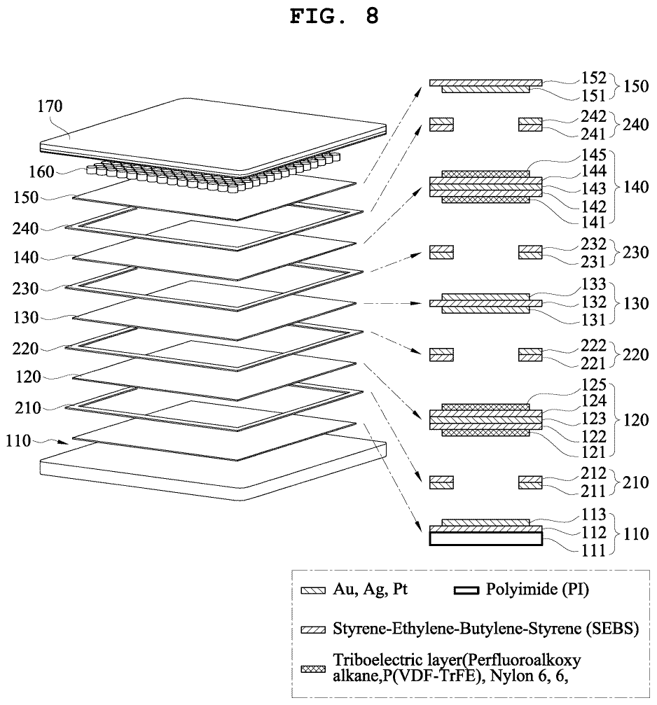

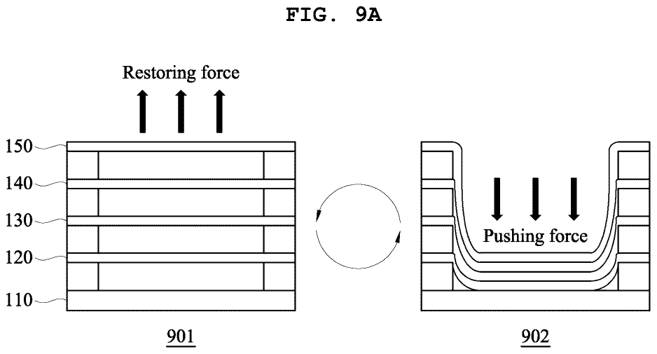

FIG. 8 is a diagram for explaining a cross-section of an implantable device according to one embodiment.

The substrate 110 may have a dielectric layer 112 and a first electrode layer 113 disposed thereon. The first electrode layer 113 may be disposed to expose at least a part of an upper portion of the dielectric layer 112. The substrate 110 may include a base 111 for supporting the dielectric layer 112 and the first electrode layer 113. The base 111 may include polyimide and/or polyethylene terephthalate (PET). The dielectric layer 112 may include styrene-ethylene-butylene-styrene (SEBS) and/or polydimethylsiloxane (PDMS). The first electrode layer 113 may contain gold, silver, and/or platinum.

The first spacer 210 may be provided between the substrate 110 and the first DMD layer 120. The first spacer 210 may include a first-first electrode layer 211 disposed to cover at least a part of an upper portion of the first electrode layer 113 and a dielectric layer 212 disposed to have a same area as that of the first-first electrode layer 211. The first-first electrode layer 211 may be in contact with the first electrode layer 113 to be electrically connected thereto. The first-first electrode layer 211 may contain gold, silver, and/or platinum. The dielectric layer 212 may contain styrene-ethylene-butylene-styrene (SEBS) and/or polydimethylsiloxane (PDMS).

The first spacer 210 may be divided into a first region and a second region. The first region of the first spacer 210 may correspond to a region in which the dielectric layer 112 is exposed by the first electrode layer 113 (e.g., each region having a same area). The second region of the first spacer 210 may correspond to a region (e.g., a cover region) in which the first electrode layer 113 is covered by the first spacer 210 (e.g., each region having a same area).

When viewed from above, the first spacer 210 may have a form of a frame with an open center. When viewed from above, the first spacer 210 may have a shape in which an empty small rectangle is present inside a large rectangle. That is, the first spacer 210 may also be referred to as a “rectangular ring.” Other spacers 220 to 240 may also have a shape substantially identical to that of the first spacer 210.

The first DMD layer 120 may include a first charged layer 121, a first membrane 122 disposed on the first charged layer 121, a second electrode layer 123 disposed on the first membrane 122, a second membrane 124 disposed on the second electrode layer 123, and a second charged layer 125 disposed on the second membrane 124. The first charged layer 121 may be provided to expose at least a part of a lower portion of the first membrane 122. The second charged layer 125 may be disposed to expose at least a part of an upper portion of the second membrane 124.

The first charged layer 121 and the second charged layer 125 may include perfluoroalkoxy alkane, P(VDF-TrFE), and/or Nylon 6,6. The first membrane 122 and the second membrane 124 may be configured to provide stable structural vibration. The first membrane 122 and the second membrane 124 may include styrene-ethylene-butylene-styrene (SEBS) and/or polydimethylsiloxane (PDMS). The electrode layer 123 may contain gold, silver, and/or platinum.

The second spacer 220 may be placed between the first DMD layer 120 and the MDM layer 130. The second spacer 220 may include a third-first electrode layer 222 disposed to cover at least a part of a lower portion of a third electrode layer 131 and a dielectric layer 221 disposed to have a same area as that of the third-first electrode layer 222. The third-first electrode layer 222 may be in contact with the third electrode layer 131 to be electrically connected thereto. A material of the second spacer 220 may be substantially identical to a material of the first spacer 210. An area and a shape of the second spacer 220 may be substantially identical to an area and a shape of the first spacer 210.

The MDM M layer 130 may include the third electrode layer 131, a third membrane 132 disposed on the third electrode layer 131, and a fourth electrode layer 133 disposed on the third membrane 132. The third membrane 132 may be disposed to have at least a part of a lower portion exposed by the third electrode layer 131, and at least a part of an upper portion exposed by the fourth electrode layer 133. The third electrode layer 131 and the fourth electrode layer 133 may contain gold, silver, and/or platinum. The third membrane 132 may include styrene-ethylene-butylene-styrene (SEBS) and/or polydimethylsiloxane (PDMS).

The third spacer 230 may be provided between the MDM layer 130 and the second DMD layer 140. The third spacer 230 may include a fourth-first electrode layer 231 disposed to cover at least a part of an upper portion of the fourth electrode layer 133 and a dielectric layer 232 disposed to have a same area as that of the fourth-first electrode layer 231. The fourth-first electrode layer 231 may be in contact with the fourth electrode layer 133 to be electrically connected thereto. A material of the third spacer 230 may be substantially identical to a material of the first spacer 210.

The second DMD layer 140 may include a third charged layer 141, a fourth membrane 142 disposed on the third charged layer 141, a fifth electrode layer 143 disposed on the fourth membrane 142, a fifth membrane 144 disposed on the fifth electrode layer 143, and a fourth charged layer 145 disposed on the fifth membrane 144. The third charged layer 141 may be provided to expose at least a part of a lower portion of the fourth membrane 142. The fourth charged layer 145 may be disposed to expose at least a part of an upper portion of the fifth membrane 144. A material of the second DMD layer 140 may be substantially identical to a material of the first DMD layer 120. For example, the third charged layer 141 and the fourth charged layer 145 may include perfluoroalkoxy alkane, P(VDF-TrFE), and/or Nylon 6,6.

The fourth spacer 240 may be placed between the second DMD layer 140 and the sealing layer 150. The fourth spacer 240 may include a sixth-first electrode layer 242 disposed to cover at least a part of a lower portion of a sixth electrode layer 151 and the dielectric layer 241 disposed to have a same area as that of the sixth-first electrode layer 242. The sixth-first electrode layer 242 may be in contact with the sixth electrode layer 151 to be electrically connected thereto. A material of the fourth spacer 240 may be substantially identical to a material of the first spacer 210.

The sealing layer 150 may include the sixth electrode layer 151 and a sixth membrane 152 disposed on the sixth electrode layer 151. The sixth electrode layer 151 may be provided to expose at least a part of a lower portion of the sixth membrane 152. The sixth electrode layer 151 may contain gold, silver, and/or platinum. The sixth membrane 152 may include styrene-ethylene-butylene-styrene (SEBS) and/or polydimethylsiloxane (PDMS).

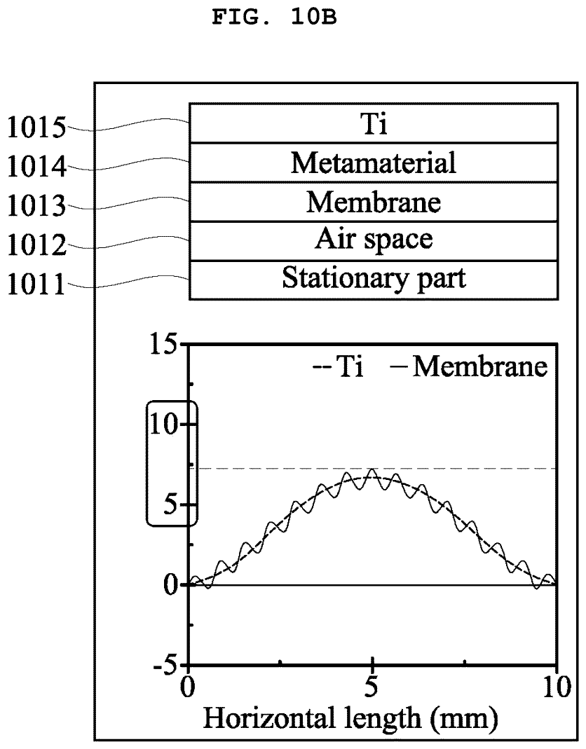

FIGS. 9A to 10B are diagrams for explaining vibration of an implantable device according to one embodiment.

Referring to FIG. 9A, vibration of an implantable device (e.g., the implantable device 100) according to one embodiment may be checked. When an ultrasound wave is applied to the implantable device (e.g., the implantable device 100), the layers 120 to 150 may vibrate relative to the substrate 110. The substrate 110 and the layers 120 to 150 may be in contact with or separate from each other. A state 901 may be a state in which the substrate 110 and the layers 120 to 150 are in a state of being separate from each other. A state 902 may be a state in which the substrate 110 and the layers 120 to 150 are in contact with each other. By alternately repeating the state 901 and the state 902, the implantable device (e.g., the implantable device 100) may vibrate.

The implantable device (e.g., the implantable device 100) according to one embodiment may include a metamaterial layer (e.g., the metamaterial layer 160). Thus, when an ultrasound wave is applied, the metamaterial layer (e.g., the metamaterial layer 160) may vibrate like a spring and efficiently apply external force to the layers 120 to 150.

As the substrate 110 and the layers 120 to 150 repeatedly come into contact with and are separated from each other according to the applied external force, friction may occur between the substrate 110 and the layers 120 to 150. This friction may induce a change in an electrical potential. That is, triboelectricity may be generated.

Referring to FIG. 9B, in a case when the substrate 110 and the layers 120 to 150 are separate from each other (e.g., a case 911), a first electrode layer (e.g., the first electrode layer 113) included in the substrate 110, a third electrode layer (e.g., the third electrode layer 131) included in the MDM layer 130, a fourth electrode layer (e.g., the fourth electrode layer 133), and a sixth electrode layer (e.g., the sixth electrode layer 151) included in a sealing layer may be changed to a negative (−) potential.

In a case when the substrate 110 and the layers 120 to 150 are separate from each other (e.g., the case 911), a second electrode layer (e.g., the second electrode layer 123) included in the first DMD layer 120, and a fifth electrode layer (e.g., the fifth electrode layer 143) included in the second DMD layer 140 may be changed to a positive (+) potential.

In a case when the substrate 110 and the layers 120 to 150 are in contact with each other (e.g. a case 912), a first electrode layer (e.g., the first electrode layer 113) included in the substrate 110, a third electrode layer included in the MDM layer 130 (e.g., the third electrode layer 131), a fourth electrode layer (e.g., the fourth electrode layer 133), and a sixth electrode layer (e.g., the sixth electrode layer 151) included in a sealing layer may be changed to a negative (−) potential.

In a case when the substrate 110 and the layers 120 to 150 are separate from each other (e.g., the case 912), a second electrode layer (e.g., the second electrode layer 123) included in the first DMD layer 120, and a fifth electrode layer (e.g., the fifth electrode layer 143) included in the second DMD layer 140 may be changed to a positive (+) potential.

In FIG. 9B, part of a dielectric layer and a membrane is not illustrated. However, it should be noted that this is for ease of the explanation.

Referring to FIG. 10A, when a membrane 1003 and a fixed portion 1002 are separate from each other only with an air layer 1002 disposed therebetween, it may be checked that a part of the membrane 1003 vibrates separately even when an ultrasound wave is applied (e.g.: multimode vibration). That is, as described above with reference to FIG. 4B, the membrane 1003 may vibrate in a multimode instead of vibrating integratedly and unidirectionally, thereby resulting in a substantial decrease in total membrane displacement.

Referring to FIG. 10B, when a metamaterial layer 1014 and a titanium layer 1015 are provided on a membrane 1013, sufficient displacement of the membrane 1013 may be secured.

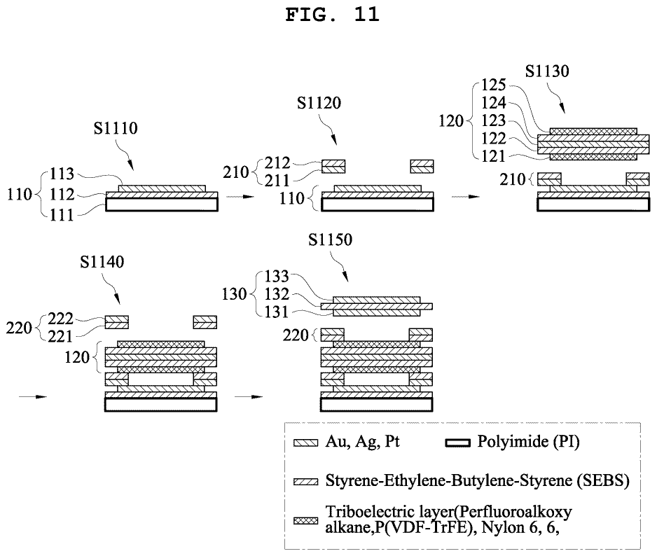

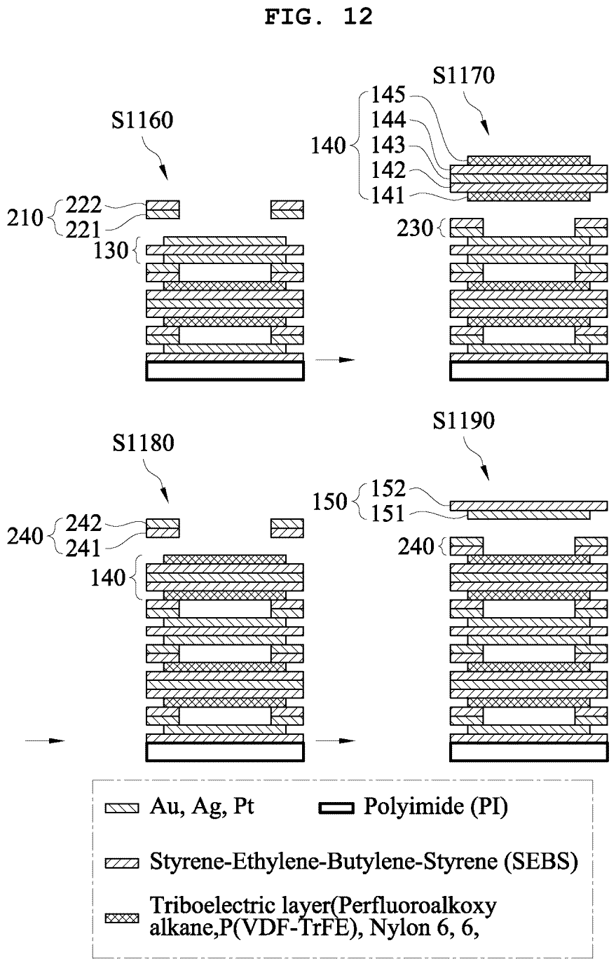

FIGS. 11 and 12 are diagrams for explaining a process of manufacturing an implantable device according to one embodiment.

In operation S1110, the substrate 110 may be provided. First, the dielectric layer 112 may be constituted by coating the flexible base 111 including polyimide or PET with a dielectric material (e.g., styrene-ethylene-butylene-styrene (SEBS) or polydimethylsiloxane (PDMS). Then, the first electrode layer 113 including gold, silver, and/or platinum may be disposed on the dielectric layer 112. The first electrode layer 113 may be disposed using a shadow mask method to expose at least a part of an upper portion of the dielectric layer 112.

In operation S1120, the first spacer 210 may be stacked on the substrate 110. The first spacer 210 may include the first-first electrode layer 211 disposed to cover at least a part of an upper portion of the first electrode layer 113 and the dielectric layer 212 disposed to have a same area as that of the first-first electrode layer 211. A method of manufacturing the first spacer 210 will be described in detail later.

When the first spacer 210 is placed on the substrate 110, the first-first electrode layer 211 may be arranged to be in contact with and electrically connected to the first electrode layer 113. To ensure stable bonding between the first electrode layer 113 and the first-first electrode layer 211, a top of the first spacer 210 may be pressed with a force of 10 N for 30 minutes. Then, likewise, in bonding between a spacer and another layer, a top thereof may also be pressed with a force of 10N for 30 minutes.

In operation S1130, the first DMD layer 120 may be stacked on the first spacer 210. In the first DMD layer 120, the first charged layer 121 may be disposed to expose at least a part of a lower portion of the first membrane 122, and the second charged layer 125 may be disposed to expose at least a part of an upper portion of the second membrane 124. A method of manufacturing the first DMD layer 120 will be described in detail later.

When the first DMD layer 120 is stacked on the first spacer 210, at least a part of an upper surface of the dielectric layer 212 may be in contact with at least a part of a lower surface of the first charged layer 121. However, since the first dielectric layer 121 is configured to have a very small thickness, the dielectric layer 212 may come in contact with the first membrane 122 depending on an embodiment.

In operation S1140, the second spacer 220 may be stacked on the first DMD layer 120. The second spacer 220 may include the dielectric layer 221 and a third-first electrode layer 222 configured to have a same area as that of the dielectric layer 221.

In operation S1150, the MDM layer 130 may be stacked on the second spacer 220. The MDM layer 130 may include the third electrode layer 131, the third membrane 132 disposed on the third electrode layer 131, and the fourth electrode layer 133 disposed on the third membrane 132. The third membrane 132 may be disposed to have at least a part of a lower portion exposed by the third electrode layer 131, and at least a part of an upper portion exposed by the fourth electrode layer 133. At this time, the third-first electrode layer 222 disposed to cover at least a part of a lower portion of the third electrode layer 131 may be in contact with the third electrode layer 131 to be electrically connected thereto.

In operation S1160, the third spacer 230 may be stacked on the MDM layer 130. The third spacer 230 may include a fourth-first electrode layer 231 disposed to cover at least a part of an upper portion of the fourth electrode layer 133 and the dielectric layer 232 disposed to have a same area as that of the fourth-first electrode layer 231. The fourth-first electrode layer 231 may be in contact with the fourth electrode layer 133 to be electrically connected thereto.

In operation S1170, the second DMD layer 140 may be stacked on the third spacer 230. The second DMD layer 140 may include the third charged layer 141, the fourth membrane 142 disposed on the third charged layer 141, the fifth electrode layer 143 disposed on the fourth membrane 142, the fifth membrane 144 disposed on the fifth electrode layer 143, and the fourth charged layer 145 disposed on the fifth membrane 144. The third charged layer 141 may be provided to expose at least a part of a lower portion of the fourth membrane 142. The fourth charged 145 may be disposed to expose at least a part of an upper portion of the fifth membrane 144.

In operation S1180, the fourth spacer 240 may be stacked on the second DMD layer 140. The fourth spacer 240 may include the dielectric layer 241 and a sixth-first electrode layer 242 configured to have a same area as that of the dielectric layer 241.

In operation S1190, the sealing layer 150 may be placed on the fourth spacer 240. The sealing layer 150 may include the sixth electrode layer 151 and a sixth membrane 152 disposed on the sixth electrode layer 151. The sixth electrode layer 151 may be provided to expose at least a part of a lower portion of the sixth membrane 152. The sixth electrode layer 151 may be in contact with the sixth-first electrode layer 242 to be electrically connected thereto.

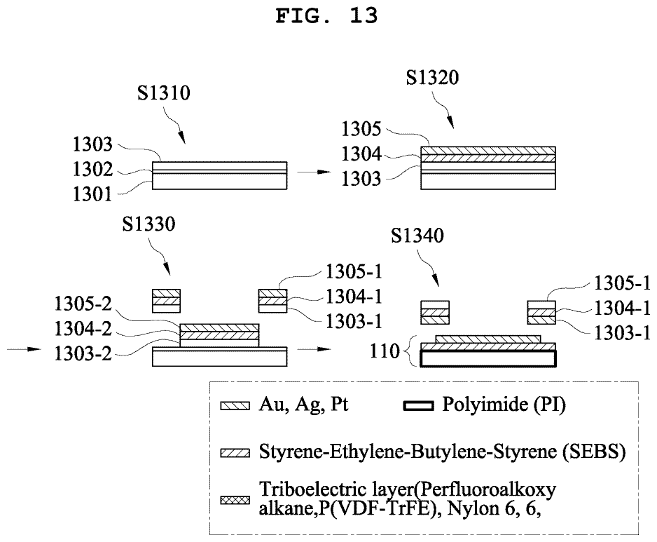

FIGS. 13 to 15 are diagrams for explaining a process of manufacturing each layer according to one embodiment.

Referring to FIG. 13, a method of manufacturing and depositing a spacer (e.g., first to fourth spacers) is shown.

In operation S1310, an adhesive may be applied to a flat substrate 1301 including glass or silicon. On an adhesive layer 1302 constituted by the applied adhesive, a non-stick coated paper layer 1303 (e.g., release paper) may be stacked. The paper layer 1303 refers to a layer specially coated on a surface so that an adhesive is not sticked to the surface, and may be coated with silicon or fluorinated compounds (PFAS).

In operation S1320, a dielectric material may be coated (e.g., bar coating or spin coating) and hardened on the paper layer 1303 to constitute a dielectric layer 1304. Thereafter, an electrode material may be deposited on the dielectric layer 1304 to constitute an electrode layer 1305.

In operation S1330, at least a part of the paper layer 1303, the dielectric layer 1304, and the electrode layer 1305 may be cut (e.g., laser cutting) to obtain first structures 1303-1 to 1305-1 and second structures 1303-2 to 1305-2. The first structures 1303-1 to 1305-1 may be configured to correspond to an area of a spacer.

In operation S1340, the first structures 1303-1 to 1305-1 may be stacked on the substrate 110, and then, the paper layer 1303 may be removed, thereby stacking the spacer on the substrate 110.

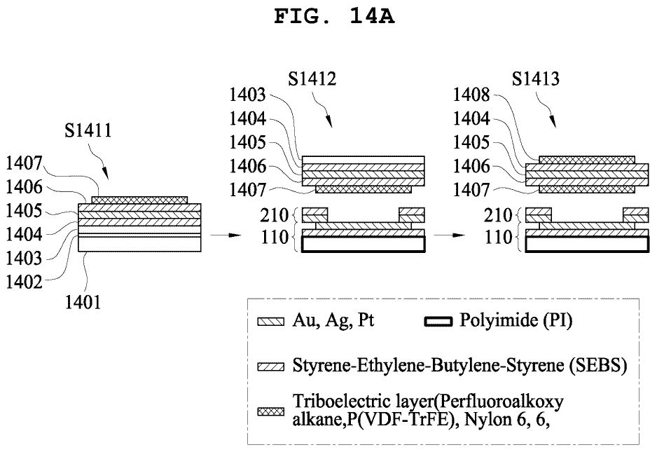

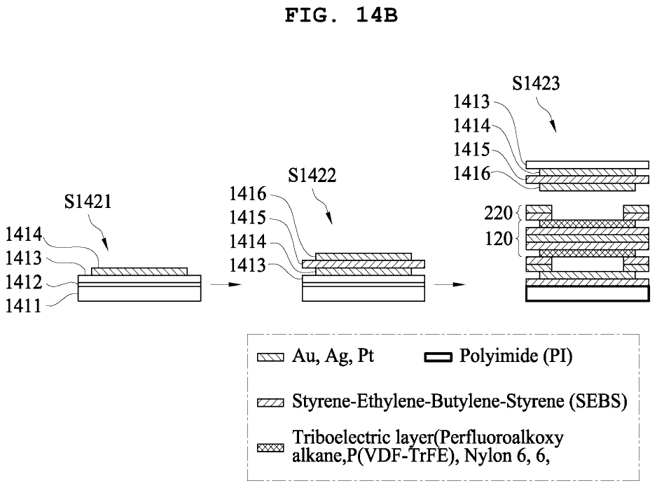

Referring to FIG. 14A, a method of manufacturing and depositing DMD layers (e.g., a first DMD layer and a second DMD layer) is shown.

In operation S1411, an adhesive may be applied to a flat substrate 1401, and a non-stick coated paper layer 1403 (e.g., release paper) may be stacked on an adhesive layer 1402 constituted by the applied adhesive. Then, a dielectric material may be coated and hardened on the paper layer 1403 (e.g., bar coating or spin coating) to constitute a dielectric layer 1404. Then, an electrode material may be deposited on the dielectric layer 1404 to constitute an electrode layer 1405. Then, a dielectric layer 1406 may be deposited on the electrode layer 1405, and a charged layer 1407 may be constituted on the dielectric layer 1406. The charged layer 1407 may be configured in a form having a coating or thin film attached thereto using a mask method to expose at least a part of an upper portion of the dielectric layer 1406.

In operation S1412, the flat substrate 1401 and the adhesive layer 1402 are removed, and the charged layer 1407 may be turned upside down to face a bottom and stacked on the first spacer 210.

In operation S1413, the paper layer 1403 may be removed, and a charged layer 1408 may be additionally disposed on the dielectric layer 1404. The charged layer 1408 may be configured in a form having a coating or thin film attached thereto using a mask method to expose at least a part of an upper portion of the dielectric layer 1404.

Referring to FIG. 14B, a method of manufacturing and depositing an MDM layer is shown.

In operation S1421, an adhesive may be applied to a flat substrate 1411, and a non-stick coated paper layer 1413 (e.g., release paper) may be stacked on the adhesive layer 1402 constituted by the applied adhesive. Then, a dielectric material may be coated (e.g., bar coating or spin coating) and hardened on the paper layer 1413 to constitute an electrode layer 1414. The electrode layer 1414 may be constituted using a shadow mask method to expose at least a part of an upper portion of the paper layer 1413.

In operation S1422, a dielectric layer 1415 may be disposed on the electrode layer 1414, and an electrode layer 1416 may be disposed on the dielectric layer 1415. The electrode layer 1416 may be constituted using a shadow mask method to expose at least a part of an upper portion of the dielectric layer 1415.

In operation S1423, the flat substrate 1411 and the adhesive layer 1412 may be removed, and the electrode layer 1415 may be turned upside down to face a bottom and stacked on the second spacer 220. Then, the paper layer 1413 may also be removed.

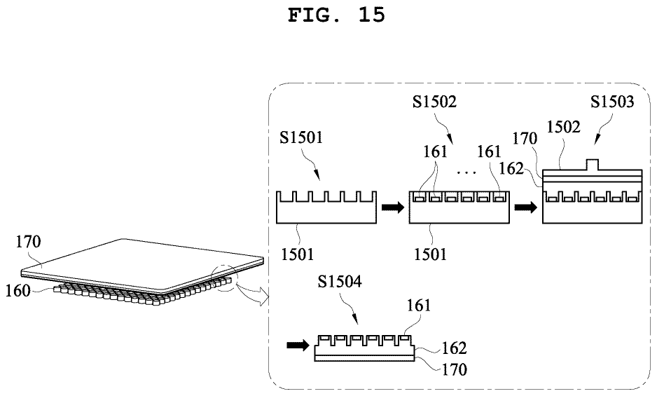

Referring to FIG. 15, a method of manufacturing the metamaterial layer 160 according to one embodiment may be identified.

In operation S1501, a mold 1501 may be prepared. The mold 1501 may include PTPE, silicone, and/or PDMS. The mold 1501 may include a plurality of wells, and the wells may function to constitute the pattern portion 162P of the metamaterial layer 160. By coating the wells included in the mold 1501 with a fluoro-octyltrichlorosilane (FOTS) self assembled monolayer (SAM), subsequent separation from the metamaterial layer 160 may be facilitated.

In operation S1502, the weight members 161 may be placed in the wells of the mold 1501, respectively. The weight members 161 may include SUS, gold, and/or platinum.

In operation S1503, a dielectric material including PDMS and/or SEBS may be coated on the mold 1501. The dielectric material may be provided in an amount sufficient to fill all of the wells of the mold 1501. Thereafter, the dielectric material may be hardened, and then, function as the elastic member 162. A titanium layer 170 may be disposed on the dielectric material, a flat plate 1502 may be brought into contact with an upper surface of the titanium layer 170, and then, the dielectric material may be hardened.

In operation S1504, the metamaterial layer 160 and the titanium layer 170 may be separated from the mold 1501.

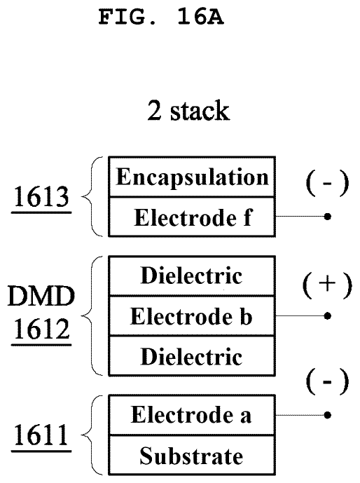

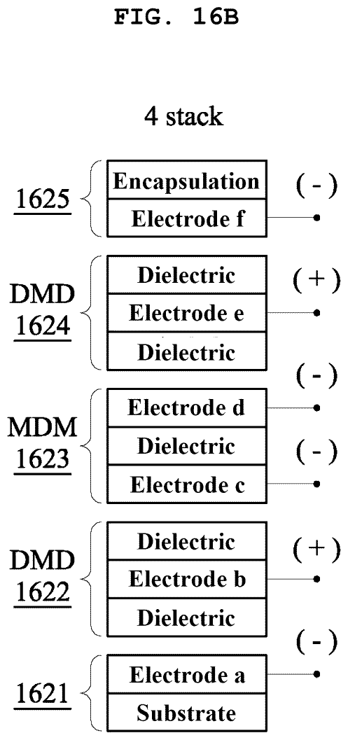

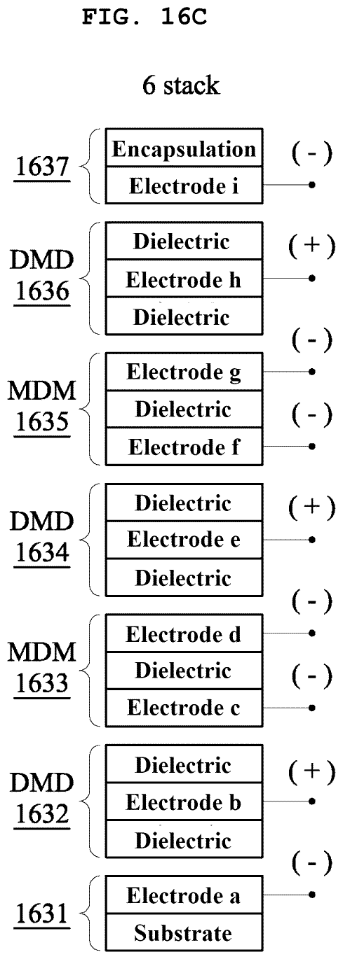

FIGS. 16A to 16C are diagrams for explaining an implantable device according to one embodiment.

Referring to FIG. 16A, an example of an implantable device implemented only using a DMD layer 1612 may be identified. The implantable device may include a substrate portion 1611, a DMD layer 1612 disposed on the substrate portion 1611, and a sealing layer 1613 disposed on the DMD layer 1612.

Referring to FIG. 16B, an example of an implantable device including two DMD layers 1622 and 1624 and one MDM layer 1623 is shown. FIG. 16B may correspond to the implantable device described with reference to this specification.

Referring to FIG. 16C, an example of an implantable device including three DMD layers 1632, 1634, and 1636 and two MDM layers 1633 and 1635 may be identified. FIG. 16B may correspond to the implantable device described with reference to this specification. In this case, when MDM layers and DMD layers have a structure symmetrical to each other, the MDM layers and the DMD layers may be sequentially alternately stacked.

While one or more embodiments of the present disclosure have been described with reference to the accompanying figures it will be understood by those of ordinary skill in the art that various changes in the form and details may be made therein without departing from the spirit and scope of the present disclosure as defined by the following claims.

The present disclosure is a submittal of research conducted under the title of “the Next-Generation Intelligent Semiconductor Technology Development R&D Program Development of MICS SoC and platform for invivo implantable electroceutical device (Project ID: 1415187321, Task Number: 20025736)” with a support of the Ministry of Trade, Industry and Energy (MOTIE) and Korea Planning & Evaluation Institute of Industrial Technology (KEIT).

The present disclosure is a submittal of research conducted under the title of “Development of a Miniaturized/High-Power Energy Solution for Battery-Free electroceutical device (project ID 2420003291, task number 00445805)” with a support of the Ministry of SMEs and Startups (MSS) and the Korea Technology & Information Promotion Agency for SMES (TIPA).

The present disclosure is a submittal of research conducted under the title of “ultrasound energy solution for remote controllable and battery replacement-free electroceutical device (DIPS 1000+) (20241755)” with a support of the Ministry of SMEs and Startups (MSS) and the Korea Institute of Startup and Entrepreneurship Development (KISED).

What is claimed is:

1. An implantable device comprising:
a substrate having a dielectric layer and a first electrode layer disposed thereon;
a first dielectric-metal-dielectric (DMD) layer disposed on the substrate and having a charged layer and a membrane disposed symmetrically to each other with reference to an electrode layer;
a metal-dielectric-metal (MDM) layer disposed on the first DMD layer and having an electrode layer symmetrically disposed with reference to the membrane;
a second dielectric-metal-dielectric (DMD) layer disposed on the MDM layer;
a sealing layer disposed on the second DMD layer; and
a metamaterial layer disposed on the sealing layer to press the sealing layer.

2. The implantable device of claim 1, wherein the metamaterial layer comprises:
weight members arranged periodically to press the sealing layer; and
an elastic member comprising a dielectric material and surrounding the weight members.

3. The implantable device of claim 2, wherein the elastic member comprises a base portion having a flat plate shape and a pattern portion protruding from the base portion toward the substrate,
the weight members are respectively provided below the pattern portion, and
the pattern portion vibrates up and down due to a weight of the weight members to expand or contract.

4. The implantable device of claim 1, further comprising:
a first spacer disposed between the substrate and the first DMD layer;
a second spacer disposed between the first DMD layer and the MDM layer;
a third spacer disposed between the MDM layer and the second DMD layer; and
a fourth spacer arranged between the second DMD layer and the sealing layer.

5. The implantable device of claim 4, wherein the first DMD layer comprises a first charged layer disposed on the first electrode layer, a first membrane disposed on the first charged layer, a second electrode layer disposed on the first membrane, a second membrane disposed on the second electrode layer, and a second charged layer disposed on the second membrane,
the first charged layer is disposed to expose at least a part of a lower portion of the first membrane, and
the second charged layer is disposed to expose at least a part of an upper portion of the second membrane.

6. The implantable device of claim 5, wherein the MDM layer comprises a third electrode layer disposed on the second charged layer, a third membrane disposed on the third electrode layer, and a fourth electrode layer disposed on the third membrane, and
the third membrane is disposed to have at least a part of a lower portion exposed by the third electrode layer and at least a part of an upper portion exposed by the fourth electrode layer.

7. The implantable device of claim 6, wherein the second DMD layer comprises a third charged layer disposed on the fourth electrode layer, a fourth membrane disposed on the third charged layer, a fifth electrode layer disposed on the fourth membrane, a fifth membrane disposed on the fifth electrode layer, and a fourth charged layer disposed on the fifth membrane,
the third charged layer is disposed to expose at least a part of a lower portion of the fourth membrane, and
the fourth charged layer is disposed to expose at least a part of an upper portion of the fifth membrane.

8. The implantable device of claim 7, wherein the sealing layer comprises a sixth electrode layer disposed on the fourth charged layer and a sixth membrane disposed on the sixth electrode layer,
the sixth electrode layer is disposed to expose at least a part of a lower portion of the sixth membrane, and
the first electrode layer is disposed to expose at least a part of an upper portion of the dielectric layer.

9. The implantable device of claim 8, wherein the first spacer comprises a first-first electrode layer disposed to cover at least a part of an upper portion of the first electrode layer and a dielectric layer disposed to have a same area as an area of the first-first electrode layer, the first-first electrode layer being in contact with and electrically connected to the first electrode layer, and
the second spacer comprises a third-first electrode layer disposed to cover at least a part of a lower portion of the third electrode layer and a dielectric layer disposed to have a same area as an area of the third-first electrode layer, the third-first electrode layer being in contact with and electrically connected to the third electrode layer.

10. The implantable device of claim 9, wherein the third spacer comprises a fourth-first electrode layer disposed to cover at least a part of an upper portion of the fourth electrode layer and a dielectric layer disposed to have a same area as an area of the fourth-first electrode layer, the fourth-first electrode layer being in contact with and electrically connected to the fourth electrode layer, and
the fourth spacer comprises a sixth-first electrode layer disposed to cover at least a part of a lower portion of the sixth electrode layer and a dielectric layer disposed to have a same area as an area of the sixth-first electrode layer, the sixth-first electrode layer being in contact with and electrically connected to the sixth electrode layer.
